(19) United States
(12) Patent Application Publication
(10) Pub. No.: US 2011/0098532 A1
(43) Pub. Date: Apr. 28, 2011

(54) METHOD FOR POSITIONING AN ENDOSCOPY CAPSULE THAT CAN BE MAGNETICALLY NAVIGATED USING A SOLENOID SYSTEM

(76) Inventors: Rainer Graumann, Hochstadt (DE); Rainer Kuth, Hochstadt (DE)

(21) Appl. No.: 12/295,043

(22) PCT Filed: Feb. 22, 2007
(86) PCT No.: PCT/EP2007/051710

§ 371 Date: Sep. 29, 2008

(30) Foreign Application Priority Data
Mar. 29, 2006 (DE) 10 2006 014 626.3

(51) Int. Cl. A61B 1/00

(52) U.S. Cl. 600/117

(57) ABSTRACT

In a method for positioning a magnetically navigable endoscopy capsule using a magnetic coil system, to bring the capsule to a known finding position within a region in the body of a patient supported on a patient table, the finding position is identified in at least one volume data set that encompasses the finding position, the volume data set is registered with respect to the patient, the volume data set and the finding position therein are transformed into the coordinate system of the magnetic coil system, and the magnetic coil system is operated to position the endoscopy capsule inserted into the patient to automatically control routing of the capsule to the finding position.

### BACKGROUND OF THE INVENTION

1. Field of the Invention

The present invention concerns a method for positioning an endoscopy capsule of the type that can be navigated magnetically by means of a magnetic coil system to conduct a medical finding in the body of a patient supported on a patient table.

2. Description of the Prior Art and Related Subject Matter

In medicine it is frequently necessary to implement a medical measure (for example a diagnosis or a treatment) internally, thus in the body of a (normally living) person or animal as a patient. The target area of such a medical measure is often a hollow organ in the appertaining patient, in particular the gastrointestinal tract. Such medical measures have been conducted with the aid of endoscopes, which are inserted into the patient from the outside through body orifices of the patient or through small incisions in a non-invasive or minimally-invasive manner, and are controlled or positioned mechanically. Inspection or manipulation devices (for example a camera or a grabber) to execute a desired action are hereby located at the tip of a more or less long, flexible catheter. Additional devices can be slid into a working channel of the catheter to the tip and be retracted again from there. Conventional endoscopes exhibit various disadvantages; for example, they cause pain or even injury in the patient due to the indirect force transfer to the catheter tip that occurs upon feeding it in, due to its length and due to the friction effects. Internal organs located farther away from the introduction site can be reached only with difficulty, or not at all.

Therefore, for a few years video capsules (from the Given Imaging corporation) that the patient swallows have been known for catheter-free or tube-free endoscopy. The video capsule moves through the alimentary canal of the patient due to peristalsis and acquires a series of video images. These are transmitted extracorporeally and stored in a recorder. The alignment of the capsule (and therefore the viewing direction of the video images) as well as the residence time in the body of the patient are random. Outside of image acquisition, the capsule has no active functionality. Diagnosis functions (such as targeted observation, cleaning, biopsies) are not possible, nor are targeted treatments inside the patient. A targeted diagnosis or finding cannot be implemented with this technology.

An endoscopy capsule that is equipped with a magnet and can move by remote control via a gradient field generated by an external magnet system is known from DE 101 42 253 C1.

A magnetic coil system that is required in order to move the magnetic endoscopy capsule through hollow organs of a patient by means of magnetic (non-contact) force transfer is described in detail in DE 103 40 925 B3. The force transfer that ensues is thus targeted, without contact and with external control. These endoscopy capsules (also called endorobots) have the functionalities of a conventional endoscope (for example video acquisition, biopsy, medicine administration etc.). A medical measure can be implemented autarchically (i.e. wirelessly or without catheter) with such an endorobot.

In German application 10 2005 032 368 (not previously published), an endorobot is described that is connected with a highly-flexible hose and draws this behind it on its path through the hollow organs, and via which treatment tasks (such as, for example, the supply of liquid or gaseous operating or working resources) can ensue, or which can be used for power supply.

Both the conventionally and mechanically positionable catheter endoscopes described above and the endorobots of the latter cited type that can be controlled without contact are controlled and moved forward manually under visual observation. The treating physician thus actively attends the entire process, from the insertion of the corresponding endoscopic apparatus into the body of the patient to its extraction from the body. This is not remarkable, since the subject of such an endoscopic examination is normally a general assessment of body regions or the search for irregularities or pathologies in these regions. In the previously unpublished German application 10 2005 007 629, a method is also specified in which the navigation of a video capsule automatically ensues along a hollow organ of a patient that forms a tube-shaped channel, wherein the video capsule advances step by step by evaluating the acquired images. The goal of this method is to provide a cost-effective and low-effort navigator that ensures a virtually gapless finding or assessment of a larger body region or organ, in particular the gastrointestinal tract.

However, in practice there is also a series of cases in which the treating physician has already diagnosed an illness or at least has a suspicion about it. This can be the result of a prior examination, for example using imaging acquisition methods that include scanning the patient, or possibly via the anamnesis or via the interview with the patient. In these cases, the approximate location of the diagnosed or suspected pathology is normally also known; the treating physician at least has an approximate idea of this location. In these cases it is desirable to move the endoscope directly and quickly into this region or to the site of interest (i.e. the finding position) in order to quickly enable the physician to implement the actual task, namely conducting detailed examination of only this region. Compared to purely reconstructive imaging methods, the use of an endoscope here can be a faster and more cost-effective method due to the photographic (and thus real) image acquisition and the combined possibility of image acquisition and parallel, active intervention possibilities. Treatment times could be minimized in this manner, and costs can therefore be saved. Today, endoscopic interventions are normally conducted by the physician. These can be time-consuming. The movement of the endoscope to the finding position requires time, even given use of an endorobot with its improved but nevertheless limited movement speed. It is desirable to provide a method that unburdens the physician from moving the endoscope to the point of interest and in this way contributes to a time savings.

### SUMMARY OF THE INVENTION

An object of the invention is to provide an improved method that enables a cost-effective, fast and nevertheless safe navigation of an endoscopy capsule in the body of the patient. It is also the object of the invention to spare the treating physician from additional work.

The object is achieved by a method for positioning a magnetically navigable endoscopy capsule via a magnetic coil system to examine a region in the body of a patient borne on a patient table, wherein the position of the examination (i.e. the finding position) is known, with the following steps:

a) identification of the finding position in at least one volume data set of the body to be examined that covers the finding position

b) registration of the volume data set to the patient

c) transformation of the volume data set and the finding position into the coordinate system of the magnetic coil system

d) positioning of the endoscopy capsule inserted into the patient at the finding position via automatic control of the magnetic coil system.

As mentioned above, the method according to the invention assumes that the treating physician already has an approximate idea of the location of the pathology or of the location of the detailed examination to be implemented. In a first method step, this location or region of interest is now labeled as a finding position in an existing volume data set of the body of the patient to be examined. The location of the finding (thus the finding position) is labeled or marked by means of an input instrument, meaning that the finding position is approached with the input instrument and an association or, respectively, linking of the finding position with the volume data set ensues. The volume data set is thus no longer or no longer exclusively used for diagnosis, but also serves as a planning aid for the use of an endoscopy capsule, in contrast to conventional procedures. In the next method step, the volume data set is registered to the patient, meaning that a correlation is established between the patient and the volume data set. The coordinates of the volume data set are mapped to the coordinates of the patient. The finding position labeled in the volume data set can then be mapped in a positionally accurate manner to the patient. In a further method step, the transformation of the volume data set and of the finding position ensues in the coordinate system of the magnetic coil system. Via this transformation it is ensured that the control of the endoscopy capsule that ensues automatically in the coordinate system of the magnetic coil system correlates with the patient position and simultaneously (in the preceding registration step) with the position of the volume data set. The finding position in the coordinate system of the magnetic coil system is then also known via this transformation step. For such a transformation step, the position of the coordinate system of the magnetic coil system and of the coordinate system of the patient or, respectively, of the volume data set relative to one another must be known. In a following method step, the endoscopy capsule inserted into the patient is automatically controlled at the site of the implementation of the finding via the activation of the magnetic coil system. The control computer of the magnetic coil system now knows the target in its coordinate system and can independently maneuver the capsule to the finding site, and the path to that site results from pre-existing route data (for example from already-stored routes), from existing volume data or, for example, by local movement control integrated into the capsule by means of image acquisition in the capsule and image processing of the currently-acquired video images.

In an embodiment, the automatic positioning is implemented using older 3D image data of the patient to be examined, contained in the volume data set. The older 3D image data can originate from a variety of 3D imaging methods (such as CT, MR, ultrasound or PET methods). In some cases, these data already exist from prior examinations or originate from earlier examinations of the patient for other illnesses.

In another embodiment, the volume data set generally contains atlas image data. Such atlas data are provided for all parts of the human body. Such anatomical atlases of the body are characterized by a high quality of the image data and are thus very well suited for planning the use of the endoscopy capsule. A first scaling of the atlas image data set (that is often sufficient for the purpose of the target positioning of the endoscopy capsule that is addressed here) ensues via the aforementioned registration procedure of the volume data set to the patient.

In a further version of this embodiment, the atlas image data are additionally scaled by at least one patient-specific value. Differences between the atlas data set and the real appearance of the patient are hereby particularly accounted for. An adaptation of the digital atlas data to the patient ensues by generating a patient-specific atlas data set from the general atlas data with the use of patient-specific varies. It has been shown that the body-mass index (BMI) of the patient, his or her height and/or his or her weight and/or his or her circumference can advantageously be used for such patient-specific scaling variables because a sufficiently precise adaptation of the general atlas data set (even of internal body regions) is possible through such general values. Such scaling values can enter into the processing of the atlas data both individually and in arbitrary combination. Such scaling methods are generally known and are used at other points in medical technology. Another scaling factor (which can likewise enter into the aforementioned scaling method alone or in combination with others) results from the surface contour of the patient. This contour can be acquired from the patient by means of laser-implemented scanning methods.

In a preferred embodiment, the registration of the volume data set to the patient ensues via at least three anatomical markers and their associated as registration points in the volume data set. In this embodiment, these typical anatomical markers of the patient also label specific bone structures in the volume data set, for example. The markers are approached or localized with the use of a pointer instrument of a position detection system. In this way an image data computer can associate the various marker points and bring the coordinate systems into congruence.

In a further embodiment, the anatomical markers are additionally labeled by artificial markers, and the artificial markers are detected by means of a position detection system used in the magnetic coil system for the position detection of the endoscopy capsule. This embodiment advantageously utilizes the fact that a position detection system (which is advantageously integrated into the magnetic coil system) is present for the position detection of the endoscopy capsule in the body of the patient. The artificial markers are designed such that they can be detected or read by just this position detection system. Here it is merely required that the detection of the artificial markers is based on the same physical principle as the position detection of the capsule itself. In the prior art cited above, position detection systems are described that are based on the electromagnetic principle in which the voltages and currents induced in the magnetic field are a measure of the position coordinates of the endoscopy capsule. Special pointer instruments can be omitted this way. The markers detected by the position detection system and the markers labeled in the volume data set can then be associated very easily and with little effort, and the image data computer is able to bring the coordinate systems into congruence.

In another embodiment, the automatic positioning of the endoscopy capsule ensues through a combination of the magnetic fields of the magnetic coil system (which magnetic fields act on the capsule) and an automatic movement of the patient table. The capsule thus can be held within the working volume of the magnetic field, which is located in the center of the magnetic coil system. Here the effect of the various magnetic fields is optimal, and therefore the movement capability of the capsule is best due to the optimal force transfer. An optimal action of the magnetic fields also means an optimal efficiency of the system and therefore also an optimal energy balance. The patient table should thereby not only move in the feed direction of the patient into the magnetic coil system (and thus in the longitudinal direction); it should also be rotatable or tiltable in any direction. The magnetic field forces can additionally be supported by gravity components to a certain extent in this manner.

### BRIEF DESCRIPTION OF THE DRAWINGS

FIG. 1 schematically illustrates an endoscopy system for making a non-invasive finding or treatment of a patient.

FIG. 2 schematically illustrates the step designated (a) of the inventive method.

FIG. 3 schematically illustrates the step designated (b) of the inventive method.

FIG. 4 is a flow chart showing the basic method's steps in an embodiment of the inventive method.

### DESCRIPTION OF THE PREFERRED EMBODIMENTS

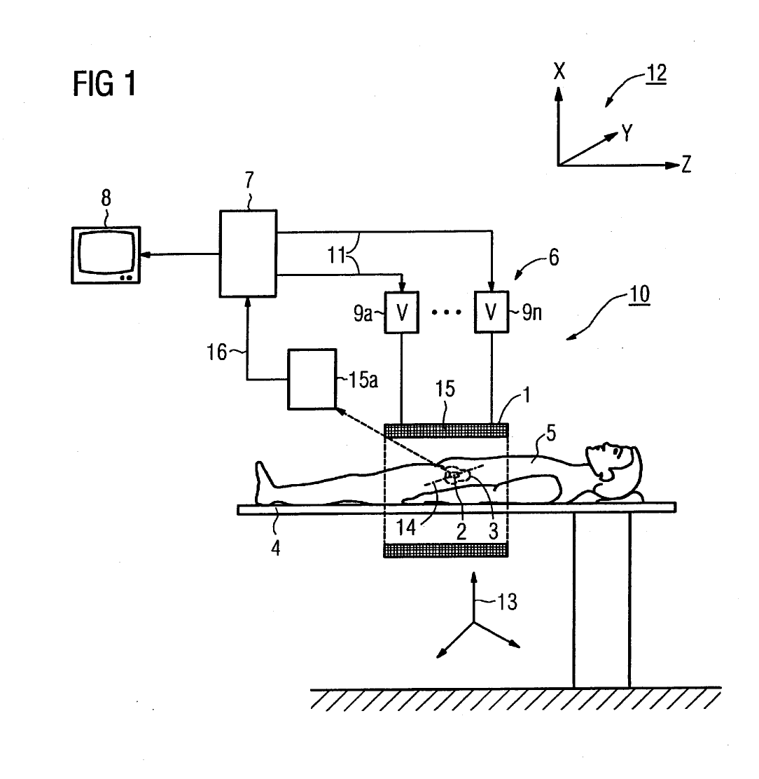

FIG. 1 shows an endoscopy system 10 for non-invasive finding or treatment of a patient 5. The endoscopy system 10 comprises a magnetic coil system 1 with a connected power supply 6 as well as a controller 7 and a display unit 8. The magnetic coil system consists of fourteen individual coils (not shown in detail here) that are divided into six rectangular Helmholtz coils arranged in a cube and eight saddle coils which together form a cuboid cylinder shell. The coil current flowing in each of the fourteen individual coils is generated by one of fourteen power amplifiers 9a-9n, each associated with an individual coil, of which only power amplifiers 9a and 9n are shown in FIG. 1. The sum of the power amplifiers 9a through 9n form the power supply 6 of the magnetic coil system 1. All power amplifiers 9a through 9n are controlled or, respectively, regulated by the controller 7 via control lines 11.

The patient 5, borne on a patient table 4, is fed into the magnetic coil system along the longitudinal axis z of the patient coordinate system 12. The patient table 4 is arbitrarily movable in the direction of the z-axis and therefore can be moved relative to the magnetic coil system 1. The patient table 4 should additionally also be arbitrarily adjustable in terms of inclination and arbitrarily pivotable if necessary, thus can move on all axes of the patient coordinate system 12.

The patient 5 is placed via the patient table 4 in the magnetic coil system 1 so that the introduced endoscopy capsule 2 is located approximately in the middle of the magnetic coil system 1. There the magnetic coil system 1 possesses what is known as the working volume. Naturally, it is also conceivable to leave the patient table 4 rigid and to be able to move the magnetic coil system 1 relative to the patient, although this variant is more complicated due to the supply lines (power supply, cooling) and the masses to be moved.

A coordinate system 13 is permanently associated with the magnetic coil system 1. The spatial position as well as the orientation of the longitudinal axis 14 of the endoscopy capsule 2 in the coordinate system 13 are determined via the position detection 15. For this it is necessary that the position detection system 15 experiences an association with the coordinate system 13. The required calibration of the position detection system 15, i.e. the determination of its relation to the magnetic coil system 1 and therefore to the coordinate system 13, ensues once upon installation of the system. For example, markers (not shown here) can thereby be attached to the coil system. The geometric alignment of the markers to the coil system is thus known, and the calibration can ensue by reading these markers via the position detection system 15, meaning that a transformation matrix between the two systems can be determined. The position detection system 15 thereby essentially consists of navigation coils and a position detection unit 15a (integrated into the magnetic coil system 1 and not labeled in detail). The spatial position as well as the orientation of the endoscopy capsule are wirelessly detected via the position detection unit 15a. The position detection unit 15a in turn transmits the position data of the endoscopy capsule via control line 16 to the controller 7. An additional magnetic field is generated by the navigation coils for the position detection of the endoscopy capsule 2, which additional magnetic field acts on position sensor coils within the endoscopy capsule 2 with one or more different frequencies and leads to the induction of same-frequency voltages and currents in the position sensor coils of the endoscopy capsule 2. These currents and voltages are then used as a position signal.

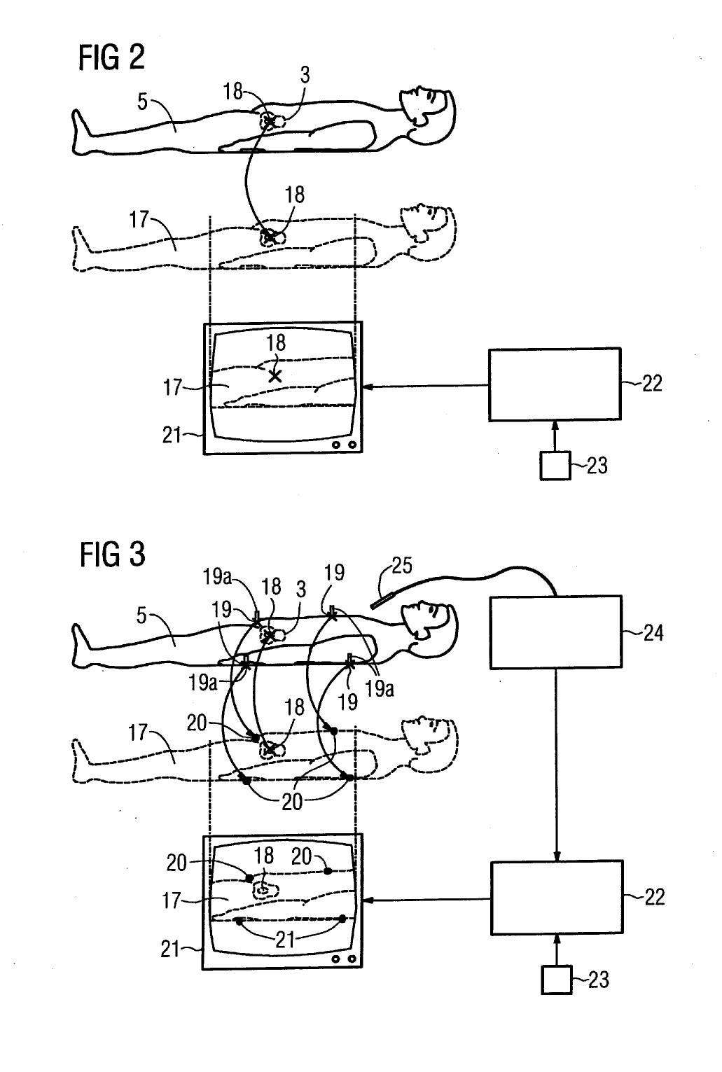

The transmission and labeling of a finding position 18 within a region 3 of the patient 5 to be examined in at least one volume data set 17 covering the finding position 18 is presented in FIG. 2. The volume data set 17 is present in suitable electronic form and is presented on a display unit 21 which is connected with a computer 22. An input instrument 23 is also connected with the computer 22. The treating physician or examiner has composed his suspicion of disease within the region 3 of the patient 5 and would like to investigate this region in more detail. The finding position 18 is marked with the input instrument 23 in a volume data set 17 (which naturally covers, among other things, the region 3 to be examined), wherein marking means a linking of the finding position 18 with or also storage in the volume data set 17. The volume data set 17 can consist of multiple older 3D images of the patient 5, acquired with any of the known methods at all. Alternatively, the volume data set can also consist of general atlas data. These are available as 3D image data in a high degree of detail for every region of the human body. A later weighting or scaling of the general atlas data with specific patient data such as BMI, height, weight, surface contour yield an additional and sufficient precision in the position determination of the target location.

FIG. 3 shows the registration of the volume data set 17 to the patient 5 for virtual labeling of the finding position 18 at the patient 5. In the exemplary embodiment, the registration ensues with prominent anatomical markers 19. At least three different markers 19 are required that need not all lie in one plane. From these markers 19, the anatomically equivalent registration points 20 are determined in the volume data set 17 and the volume data set 17 is effectively brought into congruence with the patient 5. This normally occurs via the input instrument 23 (mouse, crosshair) with which the equivalent anatomical markers 19 of the patient 5 are approached in succession as a registration point 20 in the volume data set 17 on the display unit 21 and are individually marked there in the computer 22. The markers 19 are likewise approached by a pointer instrument 25 which must be connected with a position detection or navigation system 24 which in turn interacts with the computer 22. The computer 22 can calibrate the coordinate systems of volume data set 17 and patient 5 to one another in this way. Through this procedure, the finding position 18 labeled in the volume data set 17 is also now mapped in reverse to the patient 5. In variants, artificial markers 19a are also used. These are then applied such that these artificial markers 19a additionally label the anatomical markers 19 at the patient 5 that, for example, have been localized by scanning of the patient 5 by the examiner. These artificial markers 19a can then be detected via interaction by a position detection system 24 and serve for the registration of the volume data set 17 to the patient 5. Here the position detection system 15 of the magnetic coil system 1 is advantageously used, this being necessary anyway for the control of the endoscopy capsule 2. Here it is merely necessary that the artificial markers 19a can be detected (read) by this position detection system 15, i.e. based on the same physical principle as the position detection of the capsule 2. In such a case, a pointer instrument 25 and the procedure of approaching these markers that is connected with said pointer instrument 25 can be foregone.

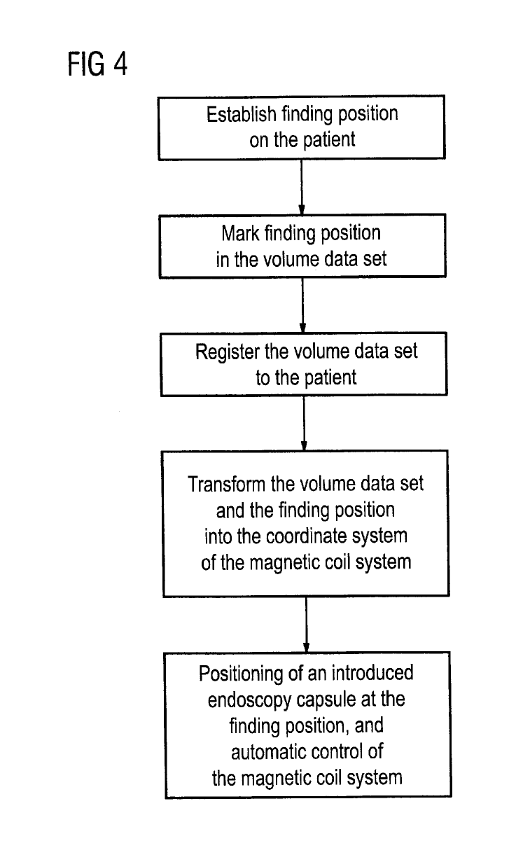

The basic method steps combined into a workflow diagram are presented in FIG. 4.

Although modifications and changes may be suggested by those skilled in the art, it is the intention of the inventors to embody within the patent warranted heron all changes and modifications as reasonably and properly come within the scope of their contribution to the art.

We claim as our invention:

1-8. (canceled)

9. A method for positioning a magnetically navigable endoscopy capsule using a magnetic coil system to bring the endoscopy capsule to a finding position in a region of a body of a patient supported on a patient table, the position of the finding position being known, comprising the steps of:
(a) identifying said finding position in at least one volume data set that encompasses the finding position;
(b) registering the volume data set relative to the patients;
(c) transforming the volume data set and the finding position therein into the coordinate system of the magnetic coil system; and
(d) positioning the endoscopy capsule in the patient at the finding position by automatically controlling the magnetic coil system dependent on the transformed volume data set and finding position in the coordinate system of the magnetic coil system.

10. A method as claimed in claim 9, comprising employing, as said volume data set, a volume data set comprising image data previously acquired from the patient.

11. A method as claimed in claim 9, comprising employing, as said volume data set, general atlas image data.

12. A method as claimed in claim 11, comprising scaling said general atlas image data dependent on at least one patient-specific variable.

13. A method as claimed in claim 12, comprising employing at least one patient-specific variable to scale said general atlas image data selected from the group consisting of body mass index of the patient, height of the patient, weight of the patient, circumference of the patient, and surface contour of the patient.

14. A method as claimed in claim 9, comprising registering the volume data relative to the patient using at least three anatomical markers and associated registration points in the volume data set.

15. A method as claimed in claim 14, comprising labeling said anatomical markers respectively with artificial markers, and detecting said artificial markers using a position detection system embodied in the magnetic coil system, for position detection of the endoscopy capsule.

16. A method as claimed in claim 9, comprising automatically positioning the endoscopy capsule in the patient using a combination of magnetic fields generated by the magnetic coil system that interact with the capsule, and automated movement of the patient table.
